(19) United States
(12) Patent Application Publication
(10) Pub. No.: US 2025/0226078 A1
(43) Pub. Date: Jul. 10, 2025

(54) SYSTEM FOR UNIFIED HEALTH AND PERFORMANCE DATA INTEGRATION

(71) Applicant: United States Performance Center, LLC, Charlotte, NC (US)

(72) Inventors: David Koerner, Waxhaw, NC (US); Irwin Belk II, Charlotte, NC (US)

(73) Assignee: United States Performance Center, LLC, Charlotte, NC (US)

(21) Appl. No.: 19/016,776

(22) Filed: Jan. 10, 2025

Related U.S. Application Data
(60) Provisional application No. 63/619,370, filed on Jan. 10, 2024.

(51) Int. Cl. G16H 20/30; G16H 10/60; G16H 40/67; G16H 50/30

(52) U.S. Cl. CPC G16H 20/30; G16H 10/60; G16H 40/67; G16H 50/30

(57) ABSTRACT

A computerized system for collecting and normalizing health and physical performance data comprises: a central data store; multiple data sources communicating via a global network, each providing data in different structured formats; normalization modules translating data related to health, wellness, performance data and the like into a central format; and an end user system. The system includes a first data source associated with an exercise machine and a second data source, each with corresponding normalization modules. These modules translate data into a central format for storage in the central data store. The end user system retrieves translated data, processes it, and provides results to users. The system may include a cloud storage for data aggregation and a customized interface for different industries. This unified approach enables comprehensive health and fitness data analysis, supporting personalized recommendations and research applications through artificial intelligence and machine learning algorithms.

### CROSS-REFERENCE TO RELATED APPLICATIONS

This application claims priority to U.S. Application No. 63/619,370, titled SYSTEM FOR COLLECTION, NORMALIZATION AND ACCESS TO UNIFIED HEALTH AND PHYSICAL PERFORMANCE INFORMATION, filed Jan. 10, 2024, which is hereby incorporated by reference in its entirety.

### FIELD OF INVENTION

The present disclosure relates to a system for collecting and processing health and physical performance data, and more particularly to a system for integrating and normalizing data from diverse sources to provide unified health and performance information both locally, regionally, nationally and internationally.

### BACKGROUND

The field of health and physical performance monitoring has seen significant advancements in recent years, with a proliferation of devices and technologies capable of collecting various types of data. These range from wearable fitness trackers and smartwatches to specialized medical devices and exercise equipment. Each of these technologies typically operates independently, collecting and storing data in its own proprietary format. They are not integrated nor allow for data modeling with aggregated data. Protocols and file formats are disparate without the means for aggregation.

While the abundance of data collection points has increased the potential for comprehensive health and performance analysis, it has also created challenges in data integration and interpretation due, at least to the large increase in data recorded. Healthcare providers, fitness professionals, and individuals often struggle to consolidate and make sense of information spread across multiple platforms and formats. This fragmentation can lead to incomplete pictures of an individual's health and physical performance, hindering effective decision-making, causing inefficiencies and duplication of efforts, and diminished personalized care. Furthermore, the lack of standardization in data collection and storage methods makes it difficult to perform large-scale analysis or research that could benefit from aggregated data across populations. This limitation hampers the development of more accurate predictive models and personalized recommendations in health and fitness.

There is a need for systems that can effectively integrate, normalize, and analyze data from diverse sources to provide a unified view of an individual's health and physical performance. Such systems could potentially enhance the ability of healthcare providers and fitness professionals to make informed decisions, while also empowering individuals with more comprehensive insights into their own health and fitness status. However, creating such integrated systems presents numerous technical challenges, including data privacy concerns, the need for robust data normalization techniques, and the development of algorithms capable of deriving meaningful insights from diverse data sets. Additionally, ensuring the accuracy and reliability of aggregated data while maintaining real-time or near-real-time processing capabilities remains a significant hurdle in this field.

The integration and aggregation of diverse health and physical performance data may provide a robust foundation for advanced artificial intelligence (AI) applications. AI algorithms may leverage this comprehensive dataset to uncover complex patterns and relationships that may not be apparent through traditional analysis methods. In some cases, machine learning models trained on aggregated data from multiple sources may offer more accurate predictions and personalized recommendations. These models may consider a wide range of factors, including exercise habits, vital signs, nutrition, and medical history, to provide holistic insights into an individual's health and fitness status. AI-powered analysis of aggregated data may also facilitate population-level studies, potentially identifying trends and risk factors across different demographics. This may lead to improved public health strategies and more targeted interventions. But this cannot be done without the system described herein since the data that is needed does not exist.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computerized system for the collection and normalization of health and physical performance data is provided. The system includes a first data source in communications with a central data store using a global communications network wherein the first datastore includes data in a first structured format. The system includes a first normalization module disposed between the first data source and the data store adapted to translate the first structured format into a central structured format and transmit a first data translated into the central structured format to the central data store. The system includes a second data source in communications with a central data store using a global communications network wherein the first datastore includes data in a second structured format. The system includes a second normalization module disposed between the second data source and the data store adapted to translate the second structured format into a central structured format and transmit a second data translated into the central structured format to the central data store. The system includes an end user system adapted to receive the first translated data and the second translated data from the central data store, update the end user system and provide processed results to an end user according to the first translated data and the second translated data.

According to other aspects of the present disclosure, the system may include one or more of the following features. The first data source may be a health care provider system. The health care provider system may include vital sign sensors and other health data. The first data source may be a physical performance or training system. The first data source may be dietary management and tracking system. The first data source may be a wearable data collection system, motion capture, medical data, biometric data and other data associated with the user.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a block diagram of a dynamic inclusive software system for collecting and processing health data, according to aspects of the present disclosure.

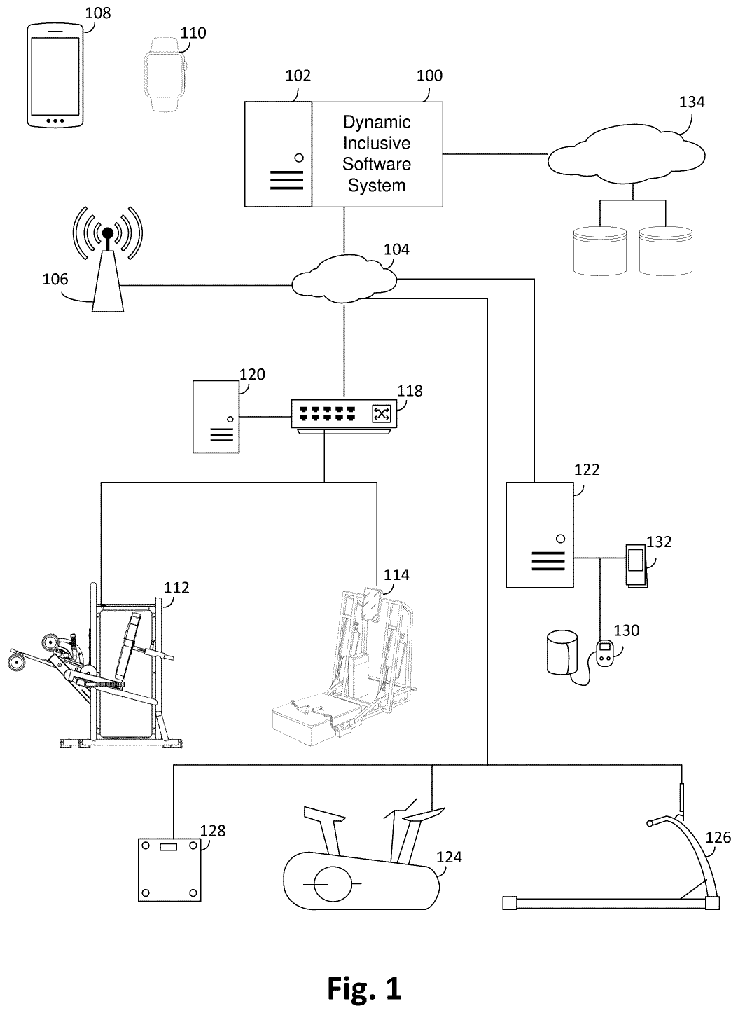

FIG. 2 illustrates a block diagram of a software system for collecting and normalizing health data, in accordance with example embodiments.

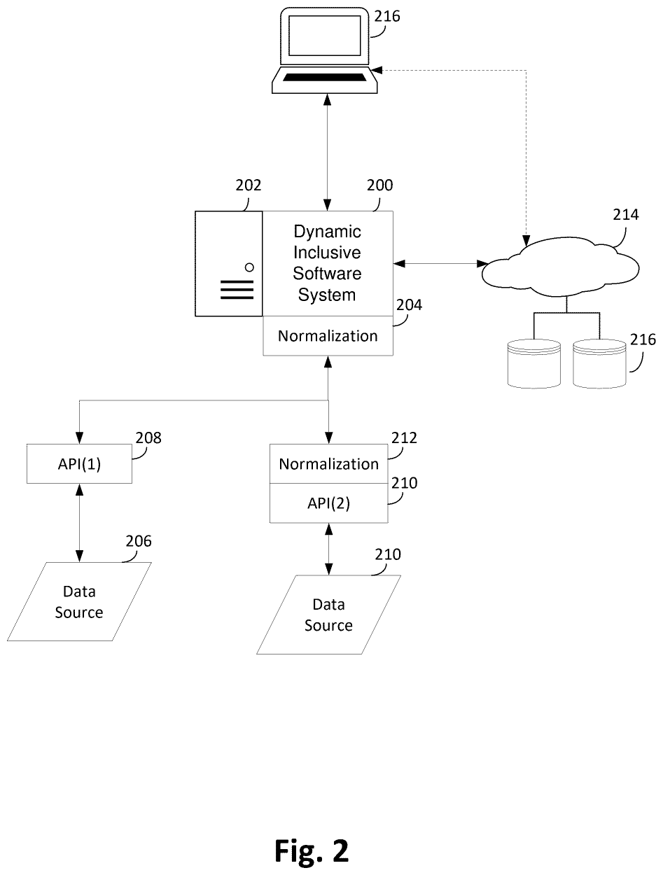

FIG. 3 illustrates a flowchart showing data flow and processing in a health and performance information system, according to an embodiment.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

This system is a computerized system for collecting and normalizing health and physical performance data from diverse sources. The system comprises multiple data sources connected to a central data store via a global communications network, with each data source having its own structured format. Normalization modules are positioned between each data source and the central data store to translate the various structured formats into a standardized central format. An end user system receives the translated data from the central data store, updates itself, and provides processed results to end users based on the aggregated data. This unified approach enables comprehensive analysis and insights by integrating data from disparate health and performance tracking systems into a single, standardized platform.

The present system offers additional advantages over existing solutions that can include enhanced data security and privacy through centralized storage and access controls, ensuring compliance with regulations like HIPAA; scalability to accommodate growing data volumes and new technologies as they emerge in the human performance field; potential for integration with wearable devices and Internet of Things (IoT) sensors, expanding the scope of data collection; improved visualization tools for complex datasets, making it easier for practitioners to interpret and communicate findings; ability to track longitudinal changes in performance metrics over time, facilitating long-term studies and personalized interventions; cost-effectiveness through the elimination of redundant data collection and analysis systems across different organizations; potential for developing machine learning models that can predict injury risks or performance outcomes with increasing accuracy over time; facilitation of remote monitoring and telehealth applications, expanding access to expert analysis and interventions; support for multi-modal data integration, combining physiological, biomechanical, and psychological metrics for a holistic view of human performance; and potential for creating standardized performance benchmarks across different populations and disciplines, advancing the field of human performance science.

The present invention may be implemented in various fields related to human performance optimization, including healthcare, sports medicine, sports performance, and research settings. By providing a centralized platform for data integration and analysis, the system can significantly improve decision-making processes and outcomes across these industries. In healthcare, the system can be utilized to enhance patient care by allowing medical professionals to access comprehensive health data from multiple sources. This integrated approach can lead to more accurate diagnoses, personalized treatment plans, and improved patient outcomes. For example, a physician could use the system to analyze a patient's medical history, current vital signs, and lifestyle data to develop a tailored treatment strategy for chronic conditions. In sports medicine and performance, the system can revolutionize athlete management and injury prevention. Coaches and trainers can leverage the platform to monitor an athlete's performance metrics, recovery data, and injury risk factors in real-time. This comprehensive view allows for timely interventions and optimized training programs, potentially reducing injury rates and enhancing overall athletic performance. Research institutions and universities can benefit from the vast database created by the system, enabling large-scale studies on human performance and health. The standardized data formats and centralized storage facilitate collaborative research efforts and the development of evidence-based practices across various disciplines. Furthermore, the system's predictive analytics and AI capabilities can be applied to identify trends and patterns in human performance data, potentially leading to groundbreaking discoveries in fields such as biomechanics, physiology, and sports science. This could result in the development of new training methodologies, rehabilitation techniques, and performance enhancement strategies. By addressing the current challenges in data collection, analysis, and interpretation, the present invention can significantly advance our understanding of human performance and drive innovation across multiple industries.

The dynamic inclusive software system 100 may be designed for collecting and processing health and physical performance data. In some cases, the software system 100 may include a server 102 connected to a network 104. The network 104 may enable communication between various components of the system. A wireless transmitter 106 may facilitate wireless connectivity for devices such as a smartphone 108 and a wearable device 110.

In some implementations, the software system 100 may integrate data from various exercise equipment. For example, a leg curl machine 112 and a deadlift machine 114 may be connected to the network 104 through a network switch 118. These exercise machines may communicate with a workout server 120, which may process and store exercise-related data generated from these machines.

Exercise machines may collect, store, and transmit data through various mechanisms and technologies. In some implementations, exercise machines may be equipped with sensors that measure different aspects of a user's workout. These sensors may include force sensors to measure the weight being lifted, motion sensors to track repetitions and movement patterns, and biometric sensors to monitor heart rate or other physiological parameters. The collected data may be temporarily stored in the machine's internal memory, stored for transmission to a more permeant storage location. This local storage may allow the machine to accumulate data throughout a workout session before transmission. In some cases, the exercise machine may have a display that shows real-time data to the user during their workout. For data transmission, exercise machines may utilize various communication protocols. In some implementations, machines may be equipped with Wi-Fi or Ethernet capabilities, allowing them to connect directly to a local network. Alternatively, some machines may use Bluetooth technology to communicate with a nearby device such as a smartphone or tablet, which can then relay the data to a central server.

In the context of the software system 100, exercise machines like the leg curl machine 112 and deadlift machine 114 may be connected to the network 104 through a network switch 118. This configuration may allow the machines to transmit data to the workout server 120. The workout server 120 may then process and store the exercise-related data, potentially aggregating information from multiple machines and users. The workout server may be one of many workout serves so that data from multiple machines, over multiple networks, at multiple locations can be aggregated. The transmission of data may occur in real-time during a workout, or it may be batched and sent at regular intervals or at the end of a session. The specific method may depend on factors such as network availability, data volume, and system design preferences. In some cases, exercise machines may also be capable of receiving data, not just transmitting it. This bidirectional communication may allow for updates to the machine's software, customization of workout programs based on user profiles, or adjustments to machine settings based on centralized training protocols.

The software system 100 may also incorporate a healthcare system 122. In some cases, the healthcare system 122 may be connected to the network 104, allowing for the integration of medical data and monitoring devices. Various fitness and health monitoring equipment may be connected to the system, including a stationary bike 124, a treadmill 126, and a scale 128. Medical monitoring devices such as a blood pressure monitor 130 and an oximeter 132 may be connected to the healthcare system 122, enabling the collection of vital health data.

In some implementations, the software system 100 may utilize cloud storage 134 for data storage and sharing capabilities across the network 104. The network 104 may serve as the central communication infrastructure, connecting all components and enabling data flow between the various devices, servers, and systems. The software system 100 may include artificial intelligence (AI) and machine learning capabilities. These capabilities may be used for analyzing human performance data, identifying correlations, predicting outcomes, and offering personalized recommendations. In some cases, the AI and machine learning components may process data collected from various sources such as the exercise equipment, medical devices, and personal monitoring devices. The AI analysis can be at the local machine lever, the workout server or data analysis can be of the aggregated data from multiple machines and workout services.

Additionally, the software system 100 may incorporate predictive analytics and individualized proprietary algorithms. These features may be used to identify trends, patterns, and potential performance and injury risk indicators. For example, data collected from the leg curl machine 112 and deadlift machine 114 may be analyzed to detect patterns that could indicate potential injury risks. Similarly, data from the blood pressure monitor 130 and oximeter 132 may be used to identify health trends and predict potential outcomes. The software system 100 may incorporate various data collection devices and communication infrastructure to gather and transmit health and physical performance data. In some cases, the server 102 may act as a central hub for receiving and processing data from multiple sources connected to the network 104. The network 104 may include both wired and wireless components to facilitate communication between different devices. In some implementations, the wireless transmitter 106 may enable wireless connectivity for portable devices such as the smartphone 108 and the wearable device 110. These devices may collect user-specific data such as activity levels, heart rate, and other biometric information.

In some implementations, the software system 100 may include communications with a smartphone application that enables users to monitor their personal health and performance data, as well as compare their metrics with others. The smartphone 108 may serve as a central interface for users to access and interact with their collected data. The smartphone application may connect to the server 102 through the network 104, allowing it to retrieve data from the central data store. Users may be able to view their personal health and performance metrics through customizable dashboards within the smartphone application. These dashboards may display real-time and historical data, including workout statistics, vital signs, and progress towards fitness goals. The application may also provide visualizations and graphs to help users better understand trends in their data over time.

In some aspects, the smartphone application may offer features for comparing a user's data with aggregated data from other users. This comparison may be anonymized to protect individual privacy while still providing valuable insights. Users may be able to compare their performance metrics with averages for their age group, fitness level, or other relevant demographics. The application may also incorporate social features, allowing users to connect with friends or join communities of individuals with similar health and fitness goals. In some cases, users may have the option to share certain metrics or achievements with their connections, fostering a sense of community and motivation. Additionally, the smartphone application may leverage the AI and machine learning capabilities of the software system 100 to provide personalized insights and recommendations based on the user's data. These recommendations may include suggested workout routines, nutrition advice, or alerts about potential health concerns based on trends in the user's data.

The smartphone application may also serve as a data collection point itself, potentially using the phone's built-in sensors to gather additional information about the user's activity levels, sleep patterns, or other relevant metrics. This data may be integrated with information from other sources to provide a more comprehensive view of the user's health and performance. In some implementations, the smartphone application may allow users to input additional data manually, such as dietary information or subjective measures of well-being. This user-inputted data may be combined with automatically collected data to provide a more holistic view of the user's health and lifestyle. The smartphone application may also facilitate communication between users and healthcare providers or fitness professionals. In some cases, users may be able to share their data directly with these professionals through the app, enabling more informed and personalized care or training recommendations.

Exercise equipment, including the leg curl machine 112 and the deadlift machine 114, may be connected to the network 104 through the network switch 118. In some cases, these machines may transmit data about user performance, including metrics such as weight lifted, repetitions, and exercise duration, to the workout server 120. The workout server 120 may then relay this information to the central server 102 for further processing and storage.

In some implementations, the software system 100 may integrate data from a wide variety of gym exercise equipment. This equipment may include, but is not limited to: cardiovascular machines such as treadmills, elliptical trainers, stationary bikes, rowing machines, and stair climbers; strength training equipment like power racks, smith machines, and cable machines; resistance machines targeting specific muscle groups, such as leg press machines, chest press machines, lat pulldown machines, and shoulder press machines; specialized machines such as hack squat machines, leg extension machines, and calf raise machines. These machines can be equipped with sensors and connectivity features that allow them to integrate with the software system 100. This integration may enable the collection and analysis of detailed workout data, including metrics such as weight used, repetitions performed, duration of exercise, force plates, velocity devices, timing gates, motion analysis devices and the like and including and even from analysis in some advanced implementations.

The healthcare system 122 may be integrated into the network 104 to provide medical data and monitoring capabilities. In some implementations, the healthcare system 122 may connect various medical devices such as the blood pressure monitor 130 and the oximeter 132 to the network 104. These devices may collect vital health data that can be transmitted to the server 102 for analysis and storage. Additional fitness equipment such as wearables, heart rate monitors, GPS devices and the like. The stationary bike 124, the treadmill 126, and the scale 128 may also be connected to the network 104. These devices may provide data on user workouts, including metrics like distance covered, calories burned, and body weight. In some cases, the software system 100 may utilize cloud storage 134 for data storage and sharing across the network 104. This may allow for seamless access to collected data from various authorized devices and locations. The software system 100 may include a normalization module that can translate data from different types of exercise machines to provide consistent data sets. For example, in some implementations, the normalization module may process data from single load machines and bilateral machines to create standardized output. This may allow for accurate comparison and analysis of user performance across different types of equipment.

In some cases, the software system 200 may include a server 202 that houses a normalization module 204 for processing data from multiple sources. The normalization module 204 may be designed to handle data in various formats and standardize it for storage and analysis. A first data source 206 may connect to the software system 200 through a first interface 208. In some implementations, the first interface 208 may serve as an API layer for data transmission. Similarly, a second data source 210 may connect to the software system 200 through a second normalization module 212. The second normalization module 212 may include its own API capabilities for data handling. The normalization module 204 may receive and process data from both the first data source 206 and the second data source 210. In some cases, the normalization module 204 may standardize the information into a consistent format. This process may allow for the integration of data from diverse sources such as the healthcare system 122, the workout server 120, and various fitness equipment like the leg curl machine 112 and the deadlift machine 114. The software system 200 may include a storage system 214 for maintaining the normalized data. In some implementations, the storage system 214 may be similar to or include the cloud storage 134. The normalized data stored in the storage system 214 may be accessed by an access device 216. The access device 216 may retrieve information from the system while also potentially sending data back through the normalization process.

In some cases, the software system 200 may provide customized user interfaces, displays, and reports for specific industries such as healthcare, therapy, and sports performance. These customized interfaces may be tailored to present the normalized data in formats that are most relevant and useful for practitioners in each field. For example, a healthcare professional using the access device 216 may see a different interface and set of reports compared to a sports performance coach accessing the same underlying data.

The architecture of the software system 200 may allow for bidirectional data flow between components. This design may create a complete data processing pipeline from source input through normalization to storage and access. For instance, data from the blood pressure monitor 130 or the oximeter 132 may be normalized, stored, and then accessed by healthcare professionals through industry-specific interfaces on the access device 216.

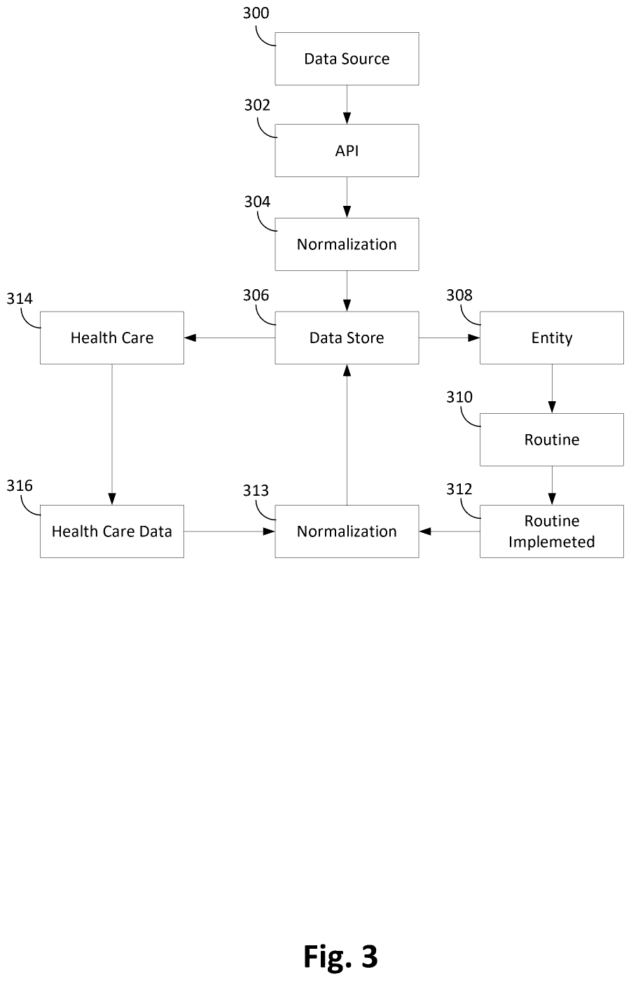

In some cases, the software system 200 may include a data flow and processing architecture as illustrated in FIG. 3. The data flow may begin with a data source module 300, which may provide raw data to an api module 302. The api module 302 may serve as an interface for receiving data from various sources, such as the smartphone 108, the wearable device 110, or the workout server 120. The data may then flow to a normalization module 304, which may process and standardize the data before sending it to a data store module 306. The normalization module 304 may perform functions similar to those of the normalization module 204, ensuring that data from diverse sources is converted into a consistent format for storage and analysis. From the data store module 306, the information may flow in two parallel paths. In one path, the data may be sent to an entity module 308. The entity module 308 may process the information according to specific entity-level requirements or permissions. For example, in some cases, the entity module 308 may filter or organize data based on the type of entity accessing the information, such as a healthcare provider or a personal trainer.

The processed data from the entity module 308 may then be sent to a routine module 310. The routine module 310 may generate specific routines or plans based on the processed data. These routines may be implemented through an implementation module 312. For instance, the implementation module 312 may execute workout routines generated based on data from the leg curl machine 112 or the deadlift machine 114. Data generated during the implementation phase may then flow to a data normalization module 313. The data normalization module 313 may standardize the implementation data before sending it back to the data store module 306, ensuring that all data in the system maintains a consistent format.

In the parallel path, data from the data store module 306 may be sent to a healthcare module 314. The healthcare module 314 may process health-related information, potentially from sources such as the blood pressure monitor 130 or the oximeter 132. The processed health data may then be sent to a healthcare data module 316, which may organize and analyze the health information. The healthcare data module 316 may then feed data to the data normalization module 313, which may normalize this healthcare information before returning it to the data store module 306. This process may ensure that health data is properly integrated with other types of performance data in the system. In some cases, the software system 200 may designate access to data at different levels. For example, the entity module 308 may control access at the entity level, allowing different types of users to access different levels of data. A healthcare provider may be granted access to more sensitive health data, while a personal trainer may have access limited to fitness-related information.

The software system 200 may also allow for different access levels to data stored in the data store module 306. In some cases, access may range from closed access, where only the user can see their own data, to open public access for certain types of aggregated or anonymized data. These access levels may be managed and enforced throughout the data flow process, ensuring that sensitive information is protected while still allowing for the sharing of relevant data among authorized users or entities.

In some cases, the software system 100 may integrate data from various sources to provide comprehensive health and performance analysis. The software system 100 may utilize data collected from devices such as the smartphone 108, wearable device 110, leg curl machine 112, deadlift machine 114, stationary bike 124, treadmill 126, scale 128, blood pressure monitor 130, and oximeter 132. This diverse range of data sources may allow for a holistic view of an individual's health and physical performance.

The normalization module 204 of the software system 200 may play a role in data integration by standardizing information from different sources into a consistent format. For example, data from the workout server 120 and the healthcare system 122 may be normalized to allow for seamless analysis and comparison. In some implementations, the data flow architecture illustrated in FIG. 3 may facilitate the utilization of integrated data. The data store module 306 may serve as a central repository for normalized data from various sources. The entity module 308 and healthcare module 314 may then process this integrated data for specific applications, such as generating personalized workout routines through the routine module 310 or analyzing health trends through the healthcare data module 316.

In one embodiment, the unified data approach of the software system 100 may offer several potential benefits. In some cases, healthcare providers may access comprehensive health and fitness data through the access device 216, allowing for more informed medical decisions. Similarly, fitness professionals may utilize integrated data to create more effective and personalized training programs. The software system 100 may also have applications in research and population health studies. In some implementations, the software system 100 may aggregate de-identified data from multiple users to create a large dataset of human performance data. This aggregated dataset may be stored in the cloud storage 134 and may serve as a valuable resource for researchers studying trends in health and physical performance across diverse populations. The ability to analyze relationships between various health and performance metrics may lead to new insights and discoveries. For example, researchers may investigate correlations between exercise habits recorded by fitness equipment like the leg curl machine 112 and health indicators measured by devices such as the blood pressure monitor 130.

In some cases, the software system 100 may employ artificial intelligence and machine learning algorithms to analyze the aggregated dataset. These advanced analytical tools may help identify patterns and trends that may not be apparent through traditional analysis methods, potentially leading to breakthroughs in understanding human health and performance. Deep learning algorithms may be particularly well-suited to handle the high-dimensional nature of aggregated health and performance data. These algorithms may discover latent features and complex interactions between variables that may inform more nuanced and personalized health recommendations. In some implementations, AI systems may use aggregated data to develop predictive models for disease progression, injury risk, or performance improvements. These models may assist healthcare providers and fitness professionals in developing proactive, data-driven strategies for patient care and athletic training. Additionally, AI-driven analysis of aggregated data may support the development of more sophisticated digital health assistants. These assistants may provide personalized guidance and support to individuals, drawing on insights derived from large-scale data analysis to offer tailored advice on lifestyle choices, exercise routines, and health management.

The system can include a software system for integrating human performance data from multiple technologies and sources. The system aims to address current challenges in data collection, analysis, and decision-making in fields such as healthcare, sports medicine, sports performance, and research. Key features include: automated data acquisition from various performance technologies; centralized data storage; customizable user experience for different industries; predictive analytics using proprietary algorithms; artificial intelligence-powered analysis; real-time data processing for timely decision-making and the creation of a comprehensive, de-identified database for research. The system can be implemented with technology companies and offered in specialized suites for different user groups. This system can streamline data management, enhance analysis capabilities, and facilitate evidence-based practices in human performance optimization across various fields.

A number of implementations have been described. Nevertheless, it will be understood that various modifications may be made without departing from the spirit and scope of the disclosure. Accordingly, other implementations are within the scope of the following claims.

What is claimed is:

1. A computerized system for collecting and normalizing health and physical performance data, comprising:
a central data store;
a first data source associated with a first exercise machine and in communication with the central data store via a global communications network, the first data source providing data in a first structured format;
a first normalization module configured to translate the first structured format into a central structured format and transmit first translated data to the central data store;
a second data source in communication with the central data store via the global communications network, the second data source providing data in a second structured format;
a second normalization module configured to translate the second structured format into the central structured format and transmit second translated data to the central data store; and
an end user system configured to receive the first translated data and the second translated data from the central data store, process the received data, and provide results to an end user based on the processed data.

2. The computerized system of claim 1, wherein the second data source is a healthcare provider system.

3. The computerized system of claim 2, wherein the healthcare provider system includes vital sign sensors.

4. The computerized system of claim 1, wherein the first data source is a physical performance system.

5. The computerized system of claim 1, wherein the end user system is associated with the physical performance system and configured to provide a customized interface for healthcare, therapy, and sports performance usages.

6. The computerized system of claim 1, further comprising a cloud storage system configured to store the first translated data and the second translated data.

7. The computerized system of claim 6, wherein the cloud storage system is further configured to aggregate de-identified data from multiple users to create an aggregated dataset.

8. A method for integrating and normalizing health and physical performance data, comprising:
receiving first data in a first structured format from a first data source wherein the first data source is a physical performance system;
translating the first data from the first structured format into a central structured format;
storing the translated first data in a central data store;
receiving second data in a second structured format from a second data source;
translating the second data from the second structured format into the central structured format;
storing the translated second data in the central data store;
retrieving the translated first data and the translated second data from the central data store;
processing the retrieved data; and
providing results based on the processed data to an end user using the physical performance system.

9. The method of claim 8, wherein the first data source is a healthcare provider system.

10. The method of claim 9, wherein the healthcare provider system includes vital sign sensors.

11. The method of claim 10, wherein the healthcare provider system includes sensors for collecting data originating from a patient.

12. The method of claim 8, further comprising:
aggregating de-identified data from multiple users to create a dataset for research purposes; and
storing the aggregated de-identified data in a cloud storage system.

13. The method of claim 12, further comprising analyzing the aggregated de-identified data using artificial intelligence and machine learning algorithms to identify patterns and trends in health and physical performance.

14. The method of claim 8, wherein processing the retrieved data comprises generating personalized workout routines based on the translated first data and the translated second data.

15. The method of claim 14, further comprising providing customized user interfaces for specific industries including healthcare, therapy, and sports performance to display the results based on the processed data.

16. A non-transitory computer-readable medium storing instructions that, when executed by a processor, cause the processor to perform operations for measuring and analyzing health and physical performance data, the operations comprising:
establishing communication with multiple data sources associated with physical performance systems via a global communications network;
receiving data from the multiple data sources in various structured formats;
normalizing the received data into a central structured format;
storing the normalized data in a central data store;
retrieving the normalized data from the central data store;
analyzing the retrieved data to generate health and performance insights; and
presenting the generated insights to an end user through a user interface.

17. The non-transitory computer-readable medium of claim 16, wherein the physical performance system is an exercise machine.

18. The non-transitory computer-readable medium of claim 17, including a healthcare provider system in communications with the central data store wherein the healthcare provider system includes vital sign sensors.

19. The non-transitory computer-readable medium of claim 16, wherein analyzing the retrieved data comprises applying machine learning algorithms to identify patterns and trends in the health and physical performance data.

20. The non-transitory computer-readable medium of claim 19, wherein the operations further comprise generating personalized recommendations based on the identified patterns and trends.
